(19) United States
(12) Patent Application Publication
(10) Pub. No.: US 2010/0076358 A1
(43) Pub. Date: Mar. 25, 2010

(54) POSTURE INDICATOR

(76) Inventor: Carolyn Anne Richardson, Warana (AU)

Correspondence Address: MARK D MILLER;KIMBLE, MACMICHAEL & UPTON, 5260 NORTH PALM AVENUE, SUITE 221, FRESNO, CA, 93704, US

(21) Appl. No.: 12/301,415

(22) PCT Filed: May 18, 2007
(86) PCT No.: PCT/AU07/00685

§ 371 Date: Nov. 18, 2008

(30) Foreign Application Priority Data
May 18, 2006 (AU) 2006902683

(51) Int. Cl. A61F 5/00

(52) U.S. Cl. 602/19

(57) ABSTRACT

The invention is directed toward an article which indicates to the wearer of the article that poor posture has been adopted.

### TECHNICAL FIELD

The invention described herein relates generally to posture. In particular, the invention is directed to an article which, with correct use: (1) indicates to a wearer of the article that good spinal posture can be achieved; (2) enhances self awareness of good spinal posture during exercise and activity; and, (3) enhances self awareness of the adoption of poor posture during exercise or activity.

### BACKGROUND ART

For an individuals health and the prevention of a range of gravity related medical conditions (including low back pain, osteoarthritis, musculoskeletal sporting injuries and stress incontinence), good posture is necessary when walking, exercising and performing the usual daily activities, especially those activities involving lifting heavy objects and bending forward for long periods. Consciously maintaining good posture during weightbearing activities, such as walking and jogging, is essential in order to activate or “turn on” the deep posture muscles which protect the joints of the body from injury and also help maintain healthy muscles and bones.

The most important part of the deep posture muscle system is the deep muscular corset which encircles the waist and supports the lower back and pelvis. This system, known as “core stability”, also involves the pelvic floor muscles, and works optimally when the spine is in its natural curves and not slumped or bent. Maintaining core stability and a healthy spine relies on self awareness of what good posture “feels” like during exercise and activity.

An antigravity postural cue (“APC”) is required to activate these muscles in weight-bearing. This cue comprises: stretching (through the back of the head) with the object of lengthening the spine; and, drawing in the navel towards the spine.

The last-mentioned action reinforces the self awareness of the natural curves of the spine while activating the deep corset and pelvic floor muscles, which narrow the waist as the “corset” tightens. However, in most individuals who have poor postural awareness and muscle weakness, these muscles can tire very quickly and so the corset is difficult to maintain. A loss of “core stability” in healthy individuals is indicated by the waist circumference increasing and the abdomen protruding. The natural curves are also lost with a bent or slumped position resulting.

From a scientific perspective (Richardson et al. 2004 Therapeutic Exercise for Lumbo-pelvic Stabilization, Churchill Livingston; Edinburgh) the many types of belts available are either for:
(1) passively supporting the low back region to assist with lifting techniques or providing comfort for low back patients;
(2) giving feedback only when gross spinal posture is changed, or waist size has increased, with no method of predicting when a user has assumed good posture (also many are sensitive, high-tech instruments which have the disadvantage of limited use in everyday exercise and activities which always involve some movement of the low back and pelvic region); or
(3) measuring levels of muscle activity of the low back directly with electromyography which is difficult and only gives some information in very controlled environments (these methods have no way of predicting when a user has assumed good posture).

Thus a major concern is that, none of the prior art can: (1) give an indication that good spinal posture has been achieved; (2) provide enhanced self awareness (via internally based sensors) of good spinal posture during exercise and activity; and, (3) provide enhanced self awareness of poor posture during exercise or activity. There is thus a need for a posture indicator which, when used appropriately during exercise and activity, allows a user to feel good posture as the waist narrows and feel poor posture through awareness of the pressure afforded by the device, when his or her waist circumference has increased and/or that the lower back has become rounded and not maintained in its natural curves.

It is an aim of the invention to provide a low-cost posture indicator which overcomes or ameliorates one or more of the disadvantages or problems described above, or which at least provides the consumer with a useful choice.

### SUMMARY OF THE INVENTION

In a first embodiment, the invention provides a posture indicator comprising:
an adjustable belt formed from a flexible but inextensible material; and
a solid and impervious spine contact member fixed to the inside surface of said belt at a portion thereof which spans the lumbar region of a wearer when said belt is in situ.

In a second embodiment, the invention provides a method of maintaining correct posture during physical activity, the method comprising:
(a) fitting a posture awareness device according to the first embodiment, wherein fitting the device comprises the steps of
   i) placing the belt of the indicator around the waist of a user so that said spine contact member rests in the lumbar curve while the user is in a standing position, and fastening the ends of the belt;
   ii) the user adopting a good postural position with the navel toward the spine while stretching upwardly through the back of the head;
   iii) the user ensuring that he or she can still breathe deeply, and
   iv) making sure that there is no pressure or tension on the belt while holding said good postural position;
(b) checking that the tension on the belt is correct by the user adopting a relaxed, slumped posture so that a tightening of the belt is felt across the abdomen and the pressure of the spine contact member against the spine is increased;
(c) if necessary, adjusting the belt to ensure that said tightening of the belt is felt across the abdomen and that the spine contact member puts pressure on the spine;
(d) commencing physical activity; and
(e) correcting posture on sensing pressure of the spine contact member on the spine and/or tightening of the belt on the abdomen.

With regard to the first embodiment of the invention defined above, the adjustable belt can be fabricated from any of the materials commonly used for belts provided that the material is inextensible. A preferred material is a woven canvas material or woven synthetic material. Belts can be provided in different lengths to allow use of the indictor by a broad range of individuals in terms of waist measurement.

The means for connecting the ends of the belt can be any suitable buckle or clasp. The means is advantageously a clasp which is adapted to permit adjustment of the length of the belt.

In a preferred embodiment, the belt is calibrated in centimetres and/or inches so that when the indicator is in situ, the waist measurement of the user is shown. The calibration can be provided by lateral marking along at least a portion of the belt or can be a separate tape which is fixed to the belt. The belt can alternatively or together with the calibration have qualitative range indicators of waist measurement such as “obese” and “normal”.

The means for joining the ends of the belt advantageously includes a window in which the waist measurement of the user of the indicator is shown. This can be part of the clasp by which the length of the belt is adjusted. So that the calibration number appears in the window, it may be necessary for the markings to be on the inside surface of the belt

The outer surface of the spine contact member—that is, the surface that contacts the lumbar region of a wearer of the indicator in the lumbar curve (that is, the low back)—is rounded so that the indicator can be comfortably worn. To afford a member that does not have sharp edges, the member is advantageously ellipsoidal in plan. However, the member can be any suitable shape.

To maximize the sensation given by the spine contact member when signaling of an incorrect posture is appropriate, the outer surface of the member can be irregular. This irregularity can be provided by transverse ridges, pebbling, or a multiplicity of protrusions from the surface of the member. The surface irregularity can be selected to suit the sensory awareness of the user. That is, a user with lower back problems may have diminished sensory awareness in the lumbar region. The outer surface of the member in such an instance can be more irregular than would be appropriate for a user with normal sensory awareness in that region.

The spine contact member can furthermore include a vibrator to enhance the sensory signal. Such a member includes a pressure switch to activate the vibrator. A mild tingling sensation from a low-voltage electric current can also be used as a sensory enhancer.

The spine contact member can be made from any suitable material but typically comprises an incompressible plastic material. The position of the member on the belt of the indicator is advantageously adjustable. This can be provided for by the surface of the member which is fixed to the belt having a loop or aperture through which the belt passes.

The posture indicator can further include an umbilicus contact member which is located on the inside of the belt in the portion of the belt that spans the umbilicus. This member is typically a flatter member than the spine contact member but is otherwise like the latter member including having irregularities on its outer surface (the surface which contacts the umbilicus of the wearer when the indicator is in situ).

The following steps are taken as a preliminary to use of the posture indicator in accordance with the second embodiment:
while in a relaxed but upright standing position, the belt of the indicator is shortened (if necessary) to fit around the waist of the user (can be over light clothing), keeping the spine contact member in the centre of the back;
the user performs the APC to adopt a good postural position: that is, drawing the navel toward the spine while stretching upwardly through the back of the head;
the user should feel the difference: i.e., “feel” good posture as the waist narrows away from the belt;
the user ensures that he or she can still breathe deeply (that is, the user must not breathe shallowly)—the belt is adjusted if necessary (lengthened slightly);
a check that the tension on the belt is correct is performed by the user:
   letting his or her body go into a relaxed, slumped posture; and
   ensuring that a tightening of the belt is felt across the abdomen and that the spine contact member puts pressure on the spine;
   (these steps indicate to the user the sensations that will be felt when deep posture muscles fatigue or the user fails to maintain the postural position cues during weight-bearing activities; if correct “feel” of the belt cannot be attained, this indicates that the individual has poor sensory awareness and would likely already have low back pain).

Use of the posture indicator during exercise not only allows correct posture to be maintained, but also ensures that the spine of the user is supported by the correct muscles.

In order that the invention may be more readily understood and put into practice, one or more preferred embodiments thereof will now be described, by way of example only, with reference to the accompanying drawings.

### BRIEF DESCRIPTION OF THE DRAWINGS

FIG. 1 is a perspective view of a posture indicator according to the invention.

FIG. 2 is an end view of the spine contact member of the indicator shown in the preceding figure.

FIG. 3 is perspective view of the umbilicus contact member of the indicator shown in FIG. 1.

FIG. 4 depicts an individual wearing the posture indicator of the invention whose posture is good.

FIG. 5 depicts an individual wearing the posture indicator of the invention whose posture is poor.

FIG. 6 depicts the role of the posture indicator of the invention during physical exertion.

### DETAILED DESCRIPTION OF PREFERRED EMBODIMENTS

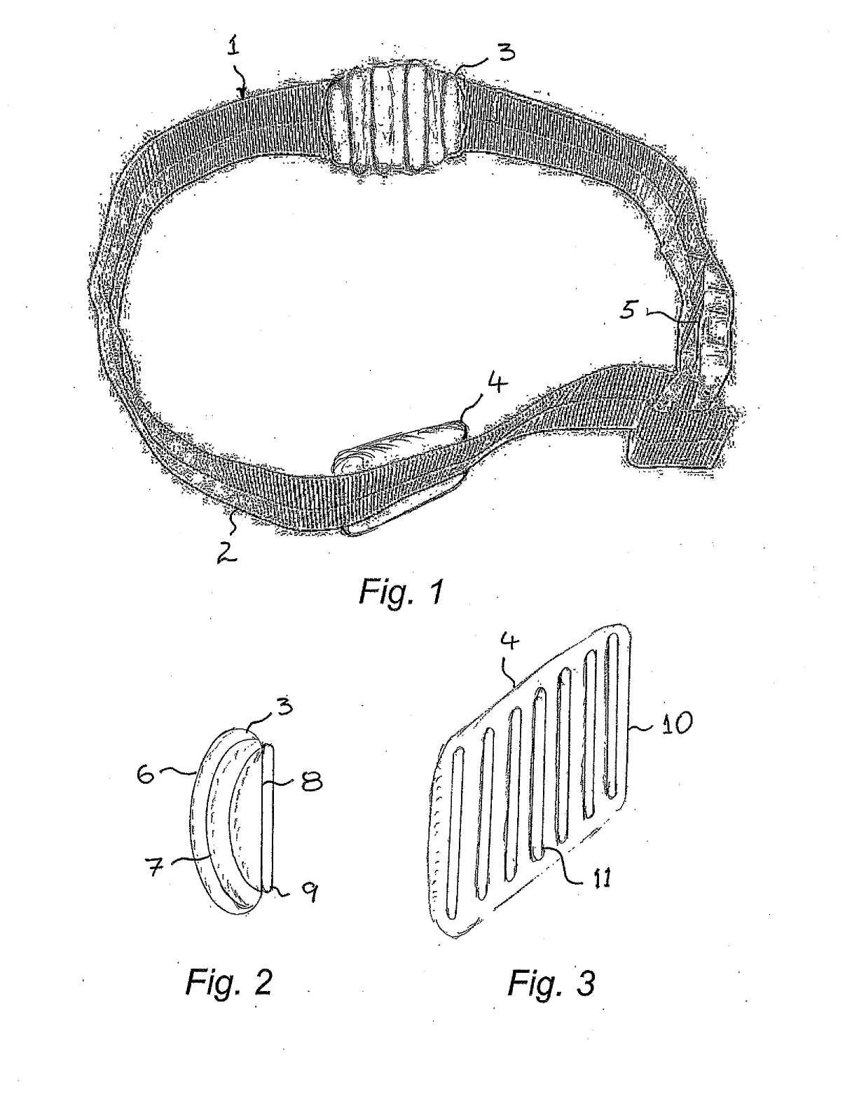

Referring firstly to FIG. 1, there is shown posture indicator 1 comprising belt 2, spine contact member 3, and umbilicus contact member 4. The belt 2 of indicator 1 has a clasp 5 which has associated therewith a belt-length adjustment mechanism. By way of this mechanism, the belt can be tensioned when in use.

The spine and umbilicus contact members 3 and 4, respectively, are shown in more detail in FIGS. 2 and 3. As shown in FIG. 2, spine contact member 3 has a curved outer surface 6. This surface also comprises a plurality of ridges, one of which is item 7. The inner surface 8 of member 3 includes a loop 9 of the same material as belt 2. Belt 2 of the indicator is passed through loop 9 which allows the position of member 3 to be adjusted in the belt when indicator 1 is in situ.

With reference to FIG. 3, there is shown umbilicus contact member 4 from which figure it can be appreciated that the member is roughly rectangular which in the same manner as spine contact member 3 has a plurality of ridges on its outer surface 10, one of which ridges is item 11. (In the foregoing context, the outer surface of the member is the surface which contacts the umbilicus.)

The surface of member 4 opposite outer surface 10 includes means for reversibly attaching the member to belt 2 of indicator 1. These means typically comprise complementary patches of hook and loop material on the member and belt.

Belt 2 of the indicator of FIG. 1 is a woven canvas material and is 5 cm wide. Spine contact member 3 of FIGS. 1 and 2 is formed from a plastics material and has dimensions of 10 cm by 7 cm by 3 cm deep. Umbilicus contact member 4 of FIGS. 1 and 3 is formed from rubber and has dimensions of 9 cm by 9 cm by 1.5 cm deep.

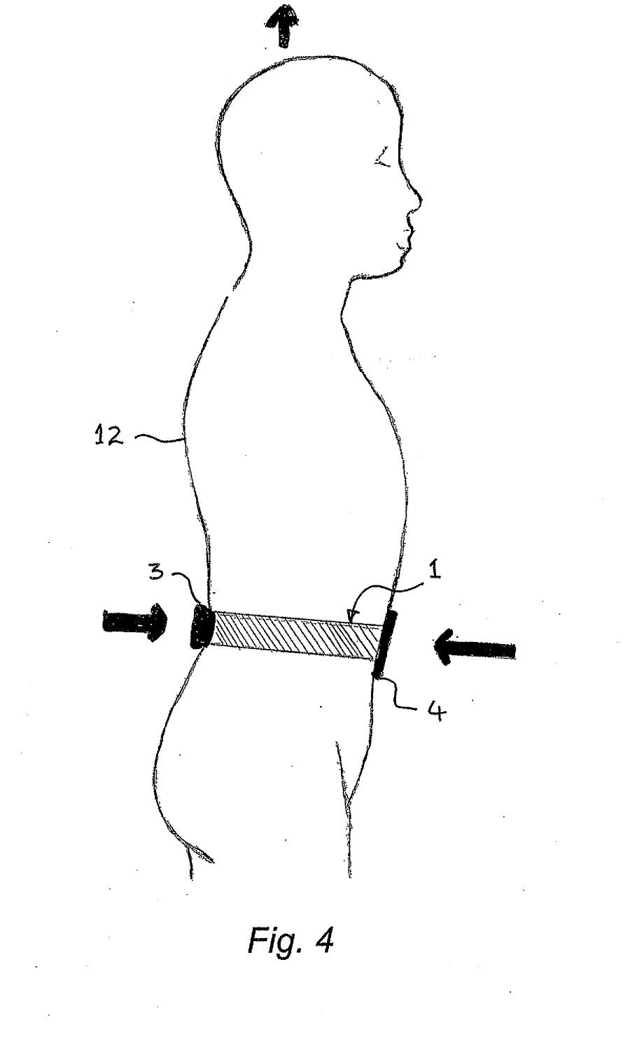

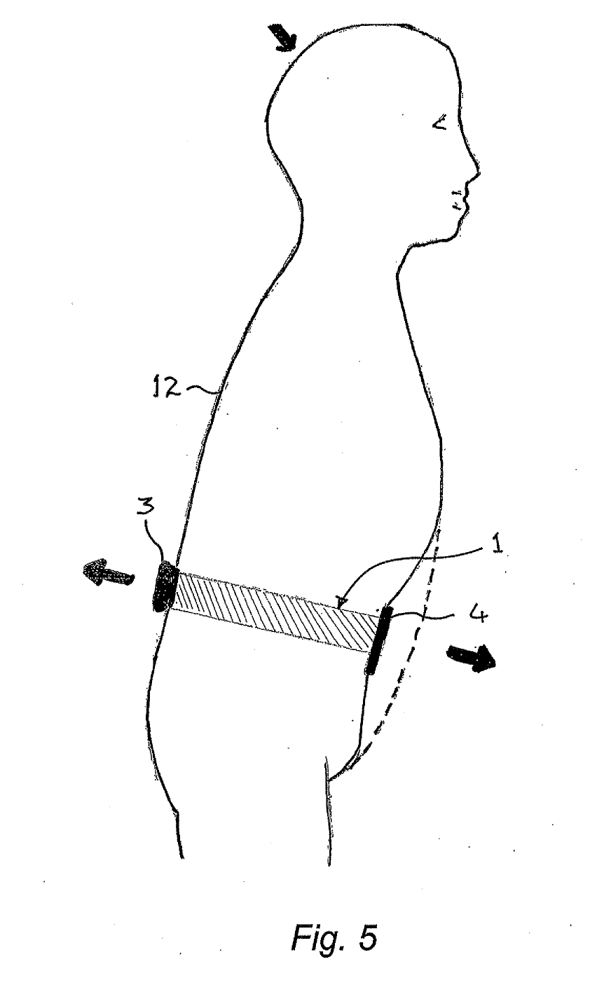

The manner in which the subject of the invention indicates good and poor posture can be appreciated from FIGS. 4 and 5. In FIG. 4, the wearer 12 of posture indicator I is standing with head extended, back straight, and stomach muscles contracted. In this position, sensations received from spine contact member 3 and umbilicus contact member 4 are minimal, as indicated by the inwardly directed arrows at the waistline of wearer 12.

An opposite situation prevails in the FIG. 5 depiction. In this instance, wearer 12 of posture indicator 1 has a slumped posture resulting in a curved back and a protruding stomach. There are thus high levels of sensation from spine contact member 3 and umbilicus contact member 4 as generally indicated by the outwardly directed arrows at the waistline of the wearer.

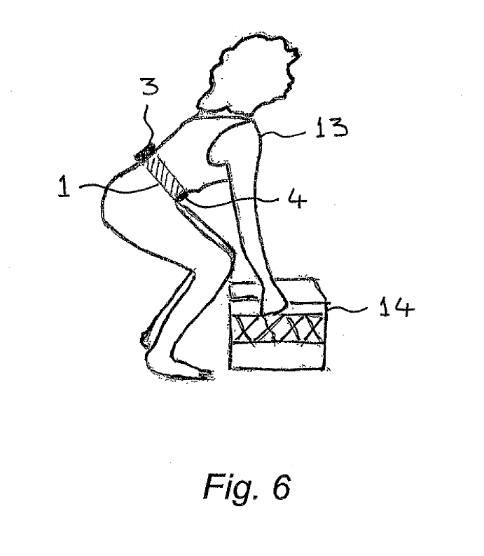

The advantages of wearing the posture indicator of the invention during physical exertion can be appreciated from FIG. 6. The sensations received from spine contact member 3 and umbilicus contact member 4 by wearer 13 of posture indicator 1 when lifting object 14 compel the wearer to adopt a straight back and to draw in the abdominal wall. The adoption of a more appropriate posture minimizes the risk of back injury.

The foregoing embodiments are illustrative only of the principles of the invention, and various modifications and changes will readily occur to those skilled in the art. The invention is capable of being practiced and carried out in various ways and in other embodiments. It is also to be understood that the terminology employed herein is for the purpose of description and should not be regarded as limiting.

The term “comprise” and variants of the term such as “comprises” or “comprising” are used herein to denote the inclusion of a stated integer or stated integers but not to exclude any other integer or any other integers, unless in the context or usage an exclusive interpretation of the term is required.

1. A posture awareness and training device comprising:
an adjustable belt formed from a flexible but inextensible material; and
a solid and impervious spine contact member comprising a plurality of ridges fixed to the inside surface of said belt at a portion thereof which spans the lumbar region of a wearer when said belt is in situ, such that in use, the spine contact member is positioned within the lumbar curve of the wearer and the ridges are in contact with the lumbar spine and adjacent lumbar regions, so as to provide tactile feedback regarding posture to the wearer without providing passive support.

2. The device of claim 1, wherein means for connecting ends of said belt comprise one of a buckle and a clasp.

3. The device of claim 2, wherein said clasp is adapted to adjustment of the length of the belt.

4. The device of claim 1, wherein said belt is calibrated in one of centimetres and inches.

5. The device of claim 4, wherein means for joining ends of said belt includes a window in which the waist measurement of the user of the indicator is shown.

6. The device of claim 1, wherein said belt has marked thereon qualitative range indicators of waist measurement.

7. The device of claim 1, wherein said spine contact member is ellipsoidal in plan.

8. The device of claim 1, wherein the outer surface of said spine contact member is irregular.

9. The device of claim 8, wherein said irregularity of the outer surface of said spine contact member is provided by one of transverse ridges, pebbling, and a multiplicity of protrusions from the surface of the member.

10. The device of claim 1, wherein the position of the spine contact member on the belt of the device is adjustable.

11. The device of claim 1, further comprising an umbilicus contact member which is located on the inside of the belt in the portion of the belt that spans the umbilicus of a user when said belt is in situ.

12. The device of claim 11, wherein the outer surface of said umbilicus contact member is irregular.

13. The device of claim 12, wherein said irregularity of the outer surface of said umbilicus contact member is provided by one of transverse ridges, pebbling, and a multiplicity of protrusions from the surface of the member.

14. A method of maintaining correct posture during physical activity, the method comprising:
(a) fitting a posture awareness device according to claim 1, wherein fitting the device comprises the steps of:
i) placing the belt of the indicator around the waist of a user so that said spine contact member rests in the lumbar curve while the user is in a standing position, and fastening the ends of the belt;
ii) the user adopting a good postural position with the navel toward the spine while stretching upwardly through the back of the head;
iii) the user ensuring that he or she can still breathe deeply; and
iv) making sure that there is no pressure or tension on the belt while holding said good postural position;
(b) checking that the tension on the belt is correct by the user adopting a relaxed, slumped posture so that a tightening of the belt is felt across the abdomen and the pressure of the spine contact member against the spine is increased;
(c) if necessary, adjusting the belt to ensure that said tightening of the belt is felt across the abdomen and that the spine contact member puts pressure on the spine;
(d) commencing physical activity; and
(e) correcting posture on sensing pressure on one of the spine contact member on the spine, and tightening of the belt on the abdomen.

15. (canceled)
